(19) United States
(12) Patent Application Publication
(10) Pub. No.: US 2002/0142288 A1
(43) Pub. Date: Oct. 3, 2002

(54) Auto-sampler for treatment or pretreatment of sample multicellular organisms for a large particle sorting flow cytometer

(76) Inventors: Peter E. Kaultkiewicz, Franklin, MA (US); John E. Memmott, Waltham, MA (US)

Correspondence Address: Choate, Hall & Stewart, Exchange Place, 53 State Street, Boston, MA, 02109, US

(21) Appl. No.: 10/077,561

(22) Filed: Feb. 15, 2002

Related U.S. Application Data
(60) Provisional application No. 60/269,503, filed on Feb. 16, 2001.

(51) Int. Cl. C12Q 1/00

(52) U.S. Cl. 435/4

(57) ABSTRACT

A system and method for analyzing the effects of a test drug on a multicellular organism is provided. The organisms which may include embryos are labeled with a cytohistochemical reagent and sorted into wells of a microtiter plate using fluorescence activated cell sorting. To each well is added a test drug. The organisms in each well are then analyzed using a auto-sampler device to determine the effect of the test drug. The organism can then be re-sorted into a fresh titer plate.

### RELATED APPLICATIONS

The present application claims priority to co-pending provisional application, U.S. Ser. No. 60/269,503, filed Feb. 16, 2001, which is incorporated herein by reference.

### BACKGROUND OF THE INVENTION

1. Area of the Art

The present invention concerns the field of sorting flow cytometers and specifically accessories for use with a large particle flow sorter designed to sort multicellular organisms.

2. Description of the Prior Art

Flow cytometers are well known analytical instruments capable of analyzing the characteristics of large numbers of particles as they pass in single file through an analysis zone. Typically the analysis is conducted optically as the particles pass through a focused laser beam although electronic volume (“Coulter” volume) as well as a number of other analyses can be conducted. Most often in modern research the analyzed particles are single cells such as blood cells or stem cells and at least some of the optical parameters measures are provided by labeled antibodies bound to the cells. In the first generation cytometers the optical measurements were displayed as a histogram (“cytogram”) which allowed the researchers to identify a number of hitherto unknown subpopulations in the analyzed cells. Second generation flow cytometers gained the ability to select members of one or more of these populations at extremely high speeds (hundreds to thousands of cells per second). Such devices are generally known as “cell sorters” or “fluorescence activated cell sorters” (e.g., FACS® a trademark of Becton Dickinson for these devices).

Clearly the ability to select cells having particular properties, as determined by antibodies or other sensitive ligands, has revolutionized cell biology and biotechnology. It is possible to select cells with certain predefined characteristics, even where such cells are extremely rare, and then to culture the cells or otherwise use them for biotechnology or “genetic engineering”. More recently cell sorters have started to be used for pharmaceutical research and “drug discover”.

Modem drug development involves “combinatorial” chemistry wherein a large number of related chemical analogs are synthesized. Usually the basic structure of the synthesized molecules is derived from information supplied by molecular modeling based on known drug molecules or receptors or other biomolecules. Once the myriad of potential drug molecules have been synthesized, they must be tested to discover which molecules show activity. Once activity has been detected there may be one or more cycles of combinatorial synthesis based on the active molecules with the goal of producing molecules with yet a higher level of activity.

In traditional drug discovery the candidate molecules were subjected to animal testing wherein the successful candidate drugs ultimately were tested on humans. With combinatorial methods the number of candidate molecules can be so large that traditional animal testing would be not only prohibitively expensive but also of such magnitude as to be politically unacceptable with the current concern for “animal rights”. In addition, the advantage of combinatorial methods is that small amounts of a great number of candidate molecules can be efficiently produced. The quantity of each candidate molecule is generally too small for traditional animal testing and amplification of the quantities would make the methods much less economical.

There has been some success in using cell sorters to screen the candidate drugs on single cells. In some cases the potential drugs are expected to influence cellular metabolism (e.g., changes in cellular Ca⁺⁺ levels) in detectable ways. A population of “target” cells are exposed to a candidate molecule. Then the exposed cells are treated with reagents expected to convert cellular response into an optical signal-reagents such a labeled antibodies or fluorescent calcium-sensitive dyes. Then the cells are analyzed with a cell sorter and the cells showing a response are selected (sorted out) for further analysis and experimentation. Sometime the process is “multi-stage”. The cell population is first sorted to provide a subpopulation of cells known to be responsive to certain molecular signals. Then after exposure to drug candidates this sensitive subpopulation is sorted again to yield the actual responding cells.

Unfortunately, a great many tests for drug candidates cannot be carried out on single cells. To see the actual drug effects it is necessary to use a multicellular organism. One of the key discoveries of cell biology in recent decades is that many pathways and functions found in mammals are also present in much simpler multicellular organisms. The nematode Caenorhabditis elegans has only a relatively small number of cells but many of the developmental pathways of mammals and other complex vertebrate organisms are present in C. elegans. Fruit fly (Drosophila melanogaster) embryos are larger and more complex and prove to model vertebrates even more closely than C. elegans. Further, D. melanogaster has long been the subject of genetic research and a large number of well-studied mutants are available. More recently the embryos of Zebrafish (Danio rerio) have been developed as ideal models for vertebrate development.

Modern combinatorial drug discovery is now using C. elegans, D. melanogaster, and D. rerio in place of traditional animal tests. These organisms are far smaller than traditional laboratory animals and, thus far at least, have not come to the notice of animal rights protesters. However, as compared to cellular methods employing cell sorters use of these multicellular organisms is laborious and painfully slow. For the test treatments the organisms must be individually selected and pipetted by hand. The treatment analyses must be individually conducted with a microscope or similar instrumentation. Although these multicellular model organisms are tiny compared to traditional laboratory animals, they are immense compared to single cells. Therefore, traditional cell sorters are unable to process these organisms. Not only are these organisms liable to clog the flow cells designed for single cells, the piezoelectric actuators that vibrate the flow stream into individual droplets in a traditional cell sorter disrupt these multicellular organisms into mush.

Recently the assignee of the present application has developed a flow sorter optimized for analysis and sorting of multicellular organisms such as C. elegans and the embryos of D. melanogaster and D. rerio. Such instruments have been marketed as the Union Biometrica COPAS™ Sorter Instrument. Briefly, these instruments operate differently than typical sorting flow cytometers. Large diameter flow cells are utilized and the system is designed to make a sequential linear optical analysis as the elongate embryo passes through a laser beam. The fluidic stability of the flow stream and the optical analysis is ensured by having the sorting process occur on a sample stream in air well below the flow cell. Rather than vibrating the stream into droplets which are then deflected to select cell containing droplets, the entire stream is aimed into a target container-say a well of a microtiter plate. Of course, the entire stream would soon cause the well to overflow. A laterally directed stream of compressed gas intersects the sample stream and diverts it to waste. When the optical detectors determine that a desired multicellular organism has passed through the flow cell, the gas stream is momentarily interrupted so that the desired multicellular organism is deposited in the microtiter plate well. Next the microtiter plate is mechanically advanced so that the next well can be filled. This process allows the rapid deposition of selected multicellular organism. This device is more fully described in U.S. patent application Ser. No. 09/378,634, filed Aug. 20, 1999 and Ser. No. 09/465,215, filed Dec. 15, 1999, which are incorporated herein by reference.

### SUMMARY OF THE INVENTION

A drug discovery/analysis system is based on a special auto-sampler that is used together with a flow analyzer/sorter capable of analyzing and sorting large elongated multicellular organisms such as embryos of Drosophila melanogaster. The sample organisms are treated with cytohistochemical reagents designed to optically differentiate various cell biological phenomena. The organisms are suspended in a special sheath reagent and passed single file through a laser beam traversing a flow cell. Fluorescence and other optical signals are detected and analyzed to provide data reflecting the biological status of the organisms.

After passing through the flow cell, the sample stream becomes a sample stream in air which stream is accurately aimed to enter the well of a microtiter plate. A switchable pressurized gas stream is used to deflect the sample stream away from the well and into a waste area. Whenever an organism having predetermined optical characteristics passes through the flow cell, the pressurized gas stream is switched off allowing the organism to be deposited in the well. The gas stream is then switched back on, the microtiter plate is mechanically indexed to bring a fresh tray into position to receive the stream, and the entire process is repeated.

In the case of Drosophila embryos the microtiter trays can be mesh bottomed to allow excess liquid to drain. A starch thickened yeast and sugar food can then be added to each well (along with a test drug sample if required in the experimental protocol) and the entire tray sealed with a gas permeable membrane and incubated to allow the embryos to grow an react to the test substances.

Finally, the tray is placed on the stage of an auto-sampler device. A movable multi-lumened probe is directed to each well in turn. The probe penetrates the sealing membrane and enters the well. Wash fluid is dispensed through one lumen and the fluid is repeatedly drawn up into a second lumen to resuspend the organism. Finally, the organism is sucked out of the well and fluidically delivered to the flow cell of the flow sorted to be reanalyzed and resorted into a fresh titer plate. Prior to sorting any of a number of cytohistochemical treatments can be used to render various cell biological states optically detectable. This process allows rapid and complex compound screening using a multicellular organism target.

### DESCRIPTION OF THE FIGURES

FIG. 1 is diagrammatic drawing of the auto-sampler arm of the present invention;

FIG. 1a shows the arm from above;

FIG. 1b shows the arm from the side.

FIG. 2 is perspective view of the auto-sampler arm and its support bracket.

FIG. 3 is diagram showing the fluidic pathways of the auto-ampler device.

### DETAILED DESCRIPTION OF THE INVENTION

The following description is provided to enable any person skilled in the art to make and use the invention and sets forth the best modes contemplated by the inventor of carrying out his invention. Various modifications, however, will remain readily apparent to those skilled in the art, since the general principles of the present invention have been defined herein specifically to provide an autosampler system for use with a multicellular organism flow sorter.

The current COPAS™ sorter/dispenser is capable of distributing C. elegans, Danao, rerio embryos or Drosophila. melanogaster embryos into the wells of a 96-well microtiter plate within 2 minutes. This allows individual organisms to be quickly deposited. Further because the device optically analyzes each organism only desired organisms with particular predetermined biological characteristics are deposited. This makes it possible to select identically staged organisms, which greatly increases the uniformity of the testing process. For combinatorial chemistry drug discovery it is possible to pre-treat the entire batch of organisms with an initiator or other agent that will potentiate the drug effect. The sorting process then selects only the properly “primed” organisms.

Finally, it is possible to use a traditional robotic auto-sampler to deposit single aliquots of test compounds—a different one in each well—so that when a preselected organism is deposited, it is immediately exposed to a test substance. Additionally, some combinatorial synthetic methods synthesize test substances on the surfaces of sub-millimeter resin beads. In such situations, it is also possible to use the large particle flow sorter to select and deposit the test beads one to a well. To allow the passage of the multicellular organisms or large resin beads, the flow cell has a diameter of 0.5 mm or larger.

The Auto-Sampler has been designed to connect/mount to the COPAS instrument in such a way that it will access microtiter plates from the stage assembly. Over a sixteen-minute period, the Auto-Sampler can gently agitate, aspirate and dispense samples from a maximum of 96 wells into the COPAS instrument for analysis. The aspiration and dispensing of each sample is by means of a syringe pump driven via a stepper motor. The aspiration probe is designed as a multi-lumen stainless steel (sst) component able to simultaneously dispense wash fluid and aspirate sample from a microtiter well.

The auto-sampler is intended in to be used in conjunction with compatible growth media and can select and dispense accurate numbers of D. melanogaster embryos at specific stages from early embryos through third instar larvae into special mesh-bottom plates. The device, furthermore, can reaccess each well so as to sip and reanalyze the contents to detect, for example, changes in developmental stage protein expression as indicated by reporter gene fluorescence expression. A growth medium has been developed that can be added to each dispensed organism to allow normal growth and development prior to reanalysis.

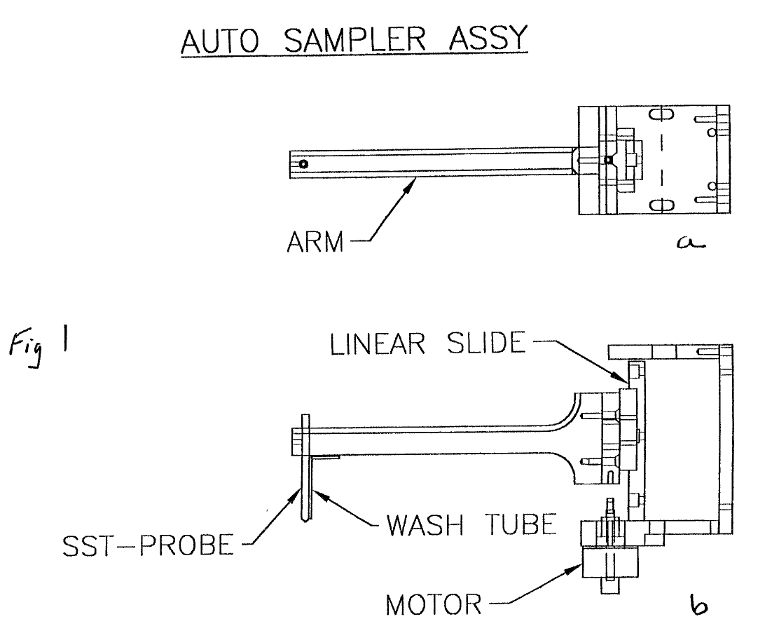

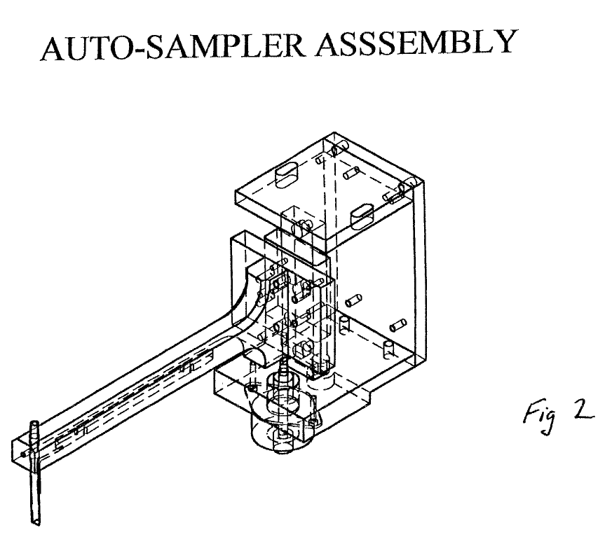

So that the reader can fully understand the present invention operation of the device during a typical experiment will now be reviewed. The auto-sampler consists of a straight arm, shown from above in FIG. 1a. As shown in FIG. 1b the sst-probe is located at a distal end of the straight arm and is connected to a syringe pump or to multiple syringe pumps (FIG. 3) by flexible tubing (not shown). The sst-probe which samples the contents of a microtiter well is joined with a parallel wash tube. The wash tube dispenses a wash solution to suspend and wash the sample prior to the aspiration by the sst-probe. The linear arm is held by a C-shaped bracket or cage (FIG. 1b, FIG. 2). The C-shaped bracket engages the arm by means of a linear slide that allows the linear arm to be driven up and down by a motor (FIG. 1b). Meanwhile, a stage supporting the microtiter plate provides x-y lateral motion of the plate relative to the probe.

In operation a microtiter plate is placed on the surface below the probe in FIG. 1b. The probe is brought into position to address any well of the plate by x-y motion of the stage relative to the probe. All of the motions are preferably provided by stepper motors so that the probe can be positioned with great precision. When the probe is exactly above a well, the entire arm moves downwards along the linear slide so that the probe can aspirate the contents of the well.

Having briefly described the operation of the probe, the entire system will now be described. The use of D. melanogaster embryos provides a convenient example. The embryos are suspended in a special viscosity enhancing reagent for the sorting process. This liquid is also used to provide the sheath fluid for hydrodynamic focusing as is well known in the art of flow cytometry. In co-owned copending PCT application PCT/US00/35543, filed on Dec. 29, 2000, which designates the United States the use of high viscosity sheath and sample reagent is described for a COPAS-type instrument. In brief, the increased viscosity of the liquid provides enhanced stability of the hydrodynamic focusing in the large bore flow cells necessary to handle D. melanogaster embryos and other multicellular organisms. Although a considerable variety of viscosity enhancing agents can be used, a preferred formulation contains 0.9% by weight polyvinyl pyrollidone (PVP) using a material with an average molecular weight of 1.3 million. In addition, the fluid contains about 0.2% by weight sodium chloride to control osmolarity along with a trace of a wetting agent such as Triton X-100 (trademark of Rohm and Haas for their brand of polyethylene glycol octylphenyl ether). Further, an effective quantity (preferably between 0.01 and 0.05% by weight) of a chelating agent such as tetrasodium EDTA (ethylenediaminetetraacetic acid) can be added as a preservative.

The subject embryos are suspended in the solution and placed in the sample container of a special flow sorter optimized to analyze and sort large elongate organisms. Prior to the analysis the embryos may be treated with any of a number of cytohistochemical dye reagents. These reagents render various parts of the embryos fluorescent depending on developmental stage, gene expression, cellular calcium level or any of a large number of developmental or cell biological factors. The suspended embryos are hydrodynamically focused and pass single file through a flow cell where a laser beam optically interrogates each embryo. The optical signals produced as the embryo passes through the beam are analyzed in a computer and based on that analysis a decision is made as to whether or not a particular embryo has the proper characteristics desired for the remainder of the experimental protocol.

After passing through the flow cell, the sample stream passes through a precision nozzle to form a stream in air. The nozzle is quite close to a microtiter plate allowing the stream to be accurately aimed into a single well of that plate. The plate is carried by an x-y indexable mechanism so that the stream can be aimed successively into each well of the plate. A switchable stream of high pressure gas strikes the sample stream in air a short distance below the nozzle. This pressurized stream deflects and disrupts the sample stream to prevent it from entering the microtiter well. However, when the computer determines that an organism having desired characteristics has passed through the flow cell, the pressurized stream is briefly turned off. This allows the organism to be deposited in the well. Then the gas stream is reactivated to prevent additional organisms or fluid from entering the well. The plate is mechanically advanced to being a new well into position and the entire sequence is repeated. In a short time each of the 96 wells contains a single embryo having preselected characteristics. In the situation of combinatorial drug discovery each well can contain a previously dispensed sample of a different test compound or the test samples can be added after further processing.

The idea of the present auto-sampler invention is to retest each organism after it has been allowed to grow for a predetermined period of time. This allows one to readily assess the effect of the test compounds. To permit growth of the D. melanogaster embryos special filter mesh bottomed titer plates are used. Plates such as Millipore Multiscreen 96 well Nylon Mesh Plates having 10 to 60 micrometer meshes (e.g., Catalog Nos. MANMN1150, MANMN2050, MANMN4050, and MANMN6050) or Millipore Multiscreen-BV 96 Well Durapore Membrane Plates (Catalog No. MABVN1210) are suitable. The mesh plates allow the excess sheath and sample liquid to drain away so that the embryos don't drown. It is also possible to rinse the deposited embryos to remove traces of sheath reagent.

After the sheath reagent is drained, a food supply is added for growth of the embryo. Generally, Drosophila embryos feed on yeast cells which grow on fermenting organic material. A preferred food mixture consists of about 3% by weight glucose, 1.5% by weight sucrose, 5% by weight corn starch and about 8% by weight baker yeast in water. The starch mixture is cooked to produce a somewhat viscous fluid. This food nourishes the embryos yet can be readily washed away by the auto-sampler to allow the embryo to be reanalyzed. Between 50 and 100 μl of the food is deposited into each well. The entire upper surface of the plate is then sealed with a gas permeable polycarbonate membrane having, for example, 5 μm pores. This allows gas exchange and prevents drying out of the embryos. The embryos are allowed to grow in an incubator for a predetermined time.

At the end of the growth phase the tray is placed on the stage of the autosampler. The entire system is controlled by a computer that already contains information about the embryo in each well. The auto-sampler operates and each well is addressed and sampled in turn. The probe penetrates the sealing membrane and wash or flush fluid is injected. FIG. 3 shows the fluid diagram of the auto-sampler. The flush solution is stored in a pressurized container. When a valve is opened, the flush solution flows into the addressed micro-titer well through one of the bores of the multi-lumened sst probe. A syringe pump connected to the other lumen of the sst-probe activates and draws fluid into and then expels fluid from the probe to gently remove the food coating and resuspend the embryo. Finally, the probe sucks the embryo from the well and delivers it to a sample storage coil. The syringe then pumps sample through the flow cell of the COPAS instrument where it is again hydrodynamically focused, analyzed and sorted into a new microtiter plate. Various kinds of sensors can be included along the tubing through which the resuspended organism travels to detect the presence of the embryo or of a fluid/air interface or some other feature.

If desired the entire growth and sorting process may be repeated. The only limit is the life cycle of the embryo which eventually pupates following a number of molts. At each stage data are captured for each embryo. Prior to analysis the embryos may be treated with cytohistochemical reagents to facilitate data collection. At each cycle different test compounds can be applied to the embryo.

It will be appreciated that a preferred means of using described liquid food/microtiter method is with the auto-sampler and the remainder of the described system. However, the combination of a removable fly food for use in micro-titer trays is itself a novel invention. Traditional mixtures to nourish the embryos cannot be removed readily. If desired various parts of the described automated method can be carried out by hand. For example, the embryos can be selected by various “manual” means (such as a light microscope) and deposited in the microtiter well. Then an aliquot of the liquid food can be added. After an appropriate growth period, the food can be washed off, either manually or automatically, and the embryo reanalyzed either manually or (as explained above) automatically.

As one of ordinary skill in the art will appreciate, this system automates the normally slow and labor intensive job of drug screening. The auto-sampler can process a well in 10-20 seconds. The sample recovery is better than 90% per well. The entire task of analysis and dispensing of test compounds can be automated. The sorting instrument selects and deposits the organisms. The optical data produced as each organism traverses the laser beam provides a wealth of information concerning gene activation and other cellular processes. It is possible to design a complex drug screen and have it rapidly carried out in a virtually automatic fashion. This system accelerates the entire drug analysis process by orders of magnitude.

What is claimed is:

1. A drug discovery/analysis system comprising a multicellular organism, a FACS sorting device, a test agent, and a auto-sampler device.

2. A method of analyzing the effect of a test agent on a multicellular organism, the method comprising steps of:
providing a multicellular organism;
sorting the organisms by FACS into wells of a microtiter plate;
adding a test agent to a well; and
analyzing the organism for an effect by the agent.

3. The method of claim 3, wherein the organism is an embryo.
